(19) United States
(12) Patent Application Publication
(10) Pub. No.: US 2012/0216806 A1
(43) Pub. Date: Aug. 30, 2012

(54) Tube Ventilated Oxygen Mask

(76) Inventors: Tracy Rookard, Fort Washington, VA (US); Charles Dawkins, Fort Wash, MD (US)

(21) Appl. No.: 13/037,123

(22) Filed: Feb. 28, 2011

(51) Int. Cl. A61M 16/06; B23P 11/00

(52) U.S. Cl. 128/203.29; 128/206.28; 29/428

(57) ABSTRACT

This invention relates to a method and system for improving oxygen flow through a standard non-rebreather oxygen mask of the type currently used in medical facilities. The standard non-rebreather oxygen mask lacks a venturi device and sometimes fails to deliver sufficient oxygenation to patients in distress, often resulting in patient intubation. The additional of upward-facing ventilation tubes attached to either side of the mask provides an accessible oxygen reservoir allowing patients access to significantly increased oxygenation with each breath, without requiring a bag assembly, power or air compression.

### CROSS REFERENCE TO RELATED APPLICATIONS

Not applicable.

### STATEMENT REGARDING FEDERALLY SPONSORED RESEARCH OR DEVELOPMENT

No federal government funds were used in researching or developing this invention.

### NAMES OF PARTIES TO A JOINT RESEARCH AGREEMENT

Not applicable.

### SEQUENCE LISTING INCLUDED AND INCORPORATED BY REFERENCE HEREIN

Not applicable.

### BACKGROUND

1. Field of the Invention

The technology relates to a method and system for improving oxygen flow through a standard non-rebreather oxygen mask of the type currently used in medical facilities. The standard non-rebreather oxygen mask typically delivers an oxygen range of 60-80% FIO2. Preventing delivery of sufficient oxygenation to patients in distress often results in patient intubation. The additional of upward-facing ventilation tubes attached to both sides of the mask provides an improved accessible oxygen reservoir allowing patients access to significantly increased oxygenation with each breath.

2. Background of the Invention

The current state of knowledge is as follows.

Oxygen masks have traditionally covered the mouth and nose of the patient, and included small ventilation holes on the sides to allow for ease of inhalation and exhalation by the patient, as disclosed in U.S. Pat. No. 4,201,205. The proximal end of the mask is usually secured to the patient by placing the strap around the patient's head, while the distal end is attached to a tube connecting the mask to an oxygen delivery source.

In most cases, the oxygen supplied to masks will first pass through a pressure regulator, used to control the high pressure of oxygen delivered from a cylinder (or other source) to a lower pressure. The oxygen flow is controlled by a flowmeter, which may be preset or selectable, and this controls the flow in a measure such as litres per minute (lpm). The typical flowmeter range for medical oxygen is between 0 and 15 lpm with some units able to obtain up to 25 liters per minute.

A wide variety of oxygen mask styles are currently available in the market. Oxygen masks are either rebreather, partial-rebreather, or non-rebreather and are defined by the amount of a patient's exhaled breath that is allowed to be re-inhaled by the patient. Oxygen masks are also differentiated by whether the mask is of the passive type, where the patient provides the breathing force, or of the positive pressure delivery type, where the patient does not provide the breathing force. The following types of oxygen supplying masks are the most commonly used in hospital settings:

The most widely used type is known as the simple or standard oxygen face mask. This oxygen mask is of the partial-rebreather type and is also a passive type of mask in that the patient is breathing of their own accord. This type of mask is typically used for non-life threatening conditions. For instance, if a patient comes into an emergency room with undiagnosed chest pains or similar symptoms, the doctor will more than likely have the patient wear a standard face mask. Depending on the fit of oxygen mask to the patient's face, the standard mask allows a certain amount of air into the mask, which then dilutes the supplied oxygen. Oxygen is supplied to the mask via a standard supply valve. Additionally, an amount of exhaled air may remain in the mask when the patient exhales, which then mixes with the room air as well as the supplied oxygen. These types of masks are known to deliver FIO2 in the range of 28% to 50%.

Another common oxygen face mask is the reservoir bag non-rebreather oxygen mask. This mask is used for patients requiring a higher level of oxygen therapy, specifically including those patients who are able to breathe but have suffered a serious loss of oxygenation. Thus, this is also a passive type oxygen mask. This mask employs a reservoir bag which gathers and stores oxygen between patient inhalations. Oxygen is supplied to a supply valve where it flows into both the reservoir bag and the mask unimpeded. The reservoir bag acts as a storage area for oxygen. This is required so that the patient may take a full breath containing mostly oxygen, an act that would not be possible without the bag.

Under normal operation the bag is filled to approximately ⅔ capacity. Upon inhaling, a portion of the gasses in the reservoir bag (approximately ⅓ of the bag volume) are inhaled by the patient. When the patient exhales, the exhalation proceeds to the environment through a plurality of perforated openings in the side of the mask. The positive pressure of the oxygen supply inhibits a large portion of the exhaled air from entering the reservoir bag, thus ensuring that proper oxygen content is present for the next breath. Strictly speaking, reservoir bag masks are partial-rebreather-type masks because a small portion of the patient's exhaled breath remains in the mask due to the physical inability to exhaust all of the mask's volume. Additionally, exhaled air may be forced into the reservoir bag. The percentage of exhaled air remaining in the mask and bag, however, is small. Masks of this type typically provide FIO2 in the range of 60% to 80%.

An additional type of commonly used masks is the bag valve mask, which is generally used for only the most distressed patients who are actively struggling to breathe. This mask is of the positive pressure delivery type because a provider must physically squeeze a bag to assist and control the patient's breathing. This mask delivers 100 percent pure oxygen and uses one-way valves to allow for exhaled air to exit the mask while also stopping exhaled air from entering the bag. As with the reservoir bag non-rebreather oxygen mask, the bag valve mask employs a bag reservoir; but, unlike the reservoir bag non-rebreather oxygen mask the bag is manually squeezed by an attendant to provide pressure and assist the patient with their breathing.

The final type of commonly occurring oxygen mask is that which utilizes a venturi device. This device acts as an entrainment device mixing oxygen and room air in a desired ratio and is a useful tool for delivering a precise level of FIO2. A venturi device increases oxygen flow through the mask and thus increases oxygenation to the patient. This device involves a large low-flow passageway from the oxygen source, followed by a reduced cross-section body which builds pressure due to a decrease in diameter. This is followed by a second, larger cross-section passageway allowing for increased oxygen flow from the high pressure release through the mask. This type of oxygen mask is also of the positive pressure delivery type in that oxygen is continuously supplied at an elevated pressure (when compared to ambient conditions).

The venturi, in this instance, acts as calibrated flow restricting device allowing precise control of the oxygen delivered to the patient. The venturi also acts to entrain a precise amount of room air into the supplied oxygen by providing a high-speed jet of oxygen which, in turn, forces a complementary volume of room air into the mask. Venturi masks are considered high-flow oxygen therapy devices because they are able to provide total inspiratory flow at a specified FIO2 to the patient. The venturi mask is usually supplied as a kit which includes multiple jets in order to set the desired FIO2. The different flow levels are usually color coded. Unfortunately, standard non-rebreather oxygen masks do not include a venturi device.

Venturi masks, although considered high flow systems, are not always able to guarantee the total flow with oxygen percentages above 35% in patients with high inspiratory flow demands. The problem with air entrainment systems is that as the FIO2 is increased, the air to oxygen ratio decreases. For example, for 30% FIO2, the ratio is 8 parts air to 1 part oxygen. For 40% FIO2, the ratio decreases to 3 to 1. Since the jets in venturi masks generally limit oxygen flow at 12 to 15 liters per minute the total flow decreases as the ratio decreases. At an oxygen flow rate of 12 liters per minute and a 30% FIO2 setting, the total flow would be 108 lpm. At a 40% FIO2 setting, the total flow would decrease to 48 lpm. As a rule of thumb, 60 lpm is considered the minimum flow rate to qualify as a high flow device.

In cases where the patient requires an oxygen flow approaching 100%, a number of devices are available. The most common is the non-rebreather mask (or reservoir mask). This mask is designed to have a minimum flow of 10 L/min. The delivered FIO2 of this system is 60-80%, depending on the oxygen flow and patient's breathing pattern.

Distressed patients suffering from hypoxia are often at a critical juncture while using an oxygen face mask. Either the mask succeeds in increasing the patient's oxygenation to a sustainable level, or fails to do so, possibly resulting in intubation of the patient. In situations where the distress occurs quickly, quick access to a mask with an oxygen reservoir system sufficient for the patient's needs can mean the difference between a patient requiring intubation or not.

Currently available non-rebreathing oxygen masks generally include a face mask portion, oxygen reservoir bag and intermediate oxygen entry port, with the oxygen reserve bag attached around, and extending away from, the distal end of the oxygen entry port. The oxygen reservoir bag is typically constructed from a thin plastic material, and is configured in the shape of a bottle having a thin neck area, a wider main body, and a shoulder portion, having a gradually increasing width, joining the neck area and the main body of the oxygen reservoir bag.

While the face mask portion of an oxygen mask is secured over the nose and mouth area of a patient using a head strap, the main body of the oxygen reservoir bag extends outwardly over the upper chest of the patient. In normal operation, sufficient oxygen should be supplied to an oxygen reservoir bag through the oxygen entry port to continuously inflate the oxygen reserve bag to at least two-thirds of its full volume. Unfortunately, as described in detail below, this operational inflation requirement and the operational orientation of the oxygen reserve bag relative to a patient's chest often results in a pinching off of the thin neck area of the oxygen reserve bag, deleteriously impeding the flow of oxygen to a patient.

U.S. Pat. No. 5,492,114 to Vroman discloses a non-rebreathing oxygen mask for supplying a continuous flow of oxygen to a patient. This patent discusses the problems of obstructed oxygen flow through non-rebreathing masks at length, and is incorporated herein by reference in its entirety. As a patient inhales wearing an oxygen mask, only a small pressure drop due to the apparatus is desirable. Patients with decreased breathing ability must be able to breathe freely so that their oxygenation is appropriate for their condition. The kinking or pinching off of the interface between the bag and the bag valve is a known problem and occurs where the patient's physical traits force the bag to not lay flat, as is required for proper use.

Problems with the other described types of oxygen masks are also well known. The bag mask and venturi mask each have their own advantages and disadvantages. The venturi mask, for example, has the disadvantage that it is unable to provide 100% oxygenation at the required airflow, thus making it of limited use during certain medical emergencies and/or procedures. The bag valve mask, though it can deliver 100% FIO2, suffers from the requirement that a human attendant must be present to squeeze the reservoir bag. This can cause problems where a facility is dealing with a multi-patient event, or otherwise has limited personnel. Patients having respiratory problems also often require inhaled medications in addition to oxygen. Such medications are most commonly administered using a device commonly known as a nebulizer. Nebulizers are powered by a low flow rate oxygen stream. In contrast, oxygen administered to patients through an oxygen mask, is usually supplied at a higher flow rate, especially in the case of patients having respiratory difficulties. Inhaled medications are packaged, and dosage amounts are determined, for use with the commonly used, low flow rate nebulizer.

An often-used practice has been to remove the mask from the patient's face and have the patient inhale the medication from the nebulizer. Because the gas stream from the nebulizer can be (if run by air) deficient in oxygen, patients will experience some discomfort due to hypoxia and consequent shortness of breath. Therefore, the patient must be periodically switched from the nebulizer to the non-rebreathing mask and then back to the nebulizer, or placing the nebulizer under the mask increasing air entrainment and decreasing FIO2. Obviously, in addition to being uncomfortable for the patient, this procedure is inconvenient and time consuming for the healthcare personnel.

U.S. Pat. No. 5,586,551 to Hilliard discloses an oxygen mask having an extended nose region to accommodate openings for both a nebulizer and the main oxygen supply depended on by the patient for adequate respiration. The mask allows the nebulizer to be used without having to remove the patient from the main oxygen supply. This patent discusses the problems of the use of nebulizers while a patient is also undergoing oxygen treatment at length, and is incorporated herein by reference in its entirety. Hilliard, suffers in that it uses a non-standard nebulizer requiring additional costly specialized non-standard equipment.

What is needed is a standard oxygen mask providing a simple and cost-effective method for increasing oxygenation for patients in distress without relying solely on a bag valve mask system when a patient has spontaneous breathing, as well as a cost-effective nebulizer attachment pathway that does not impede the free flow of oxygen from the entry port and a bag attachment means that allows for the bag to be placed in varying positions relative to the mask.

### BRIEF SUMMARY OF THE INVENTION

The novel component of the invention is the attachment of tusk-shaped reservoir tubes to both sides of an existing non-rebreather oxygen face mask, providing a permanent oxygen reservoir space which will automatically fill with excess oxygen from the O2 source during exhalations.

A second novel component of the invention is the use of a side port in an existing oxygen face mask for attachment of a nebulizer.

A third novel component of the invention is the use of an articulated swivel or pivoting connection between the reservoir bag and the oxygen delivery port to ensure proper airflow to the patient.

Certain preferred configurations and uses are described below. The present invention is not limited to these particular configurations and uses.

The present invention can be used to sufficiently oxygenate distressed or hypoxic patients such that intubation is not required, and also provide mask access to a nebulizer without risk of impeding the oxygen flow from the source.

In a preferred embodiment, an oxygen mask of the type comprising a face mask for enclosing the nose and mouth of a person, comprising the addition of one or two apertures on one or both the sides of the mask, and the insertion or attachment of a reservoir in or over each such aperture, such that the reservoirs produce a capacity to store oxygen flowing from the oxygen entry port during times that the patient is not actively inhaling.

In another preferred embodiment, the mask of claim 1, such mask wherein the apertures are circular.

In another preferred embodiment, such mask wherein the apertures are cut into either side of the nose-covering portion of the mask.

In another preferred embodiment, such mask wherein the reservoirs are tubular in shape with open ends.

In another preferred embodiment, such mask wherein the reservoir tubes are bendable and configured in an upward-facing position.

In another preferred embodiment, such mask wherein the face mask and the reservoirs are constructed of a pliable material, including without limitation plastic, silicon or rubber, or any combination thereof.

In another preferred embodiment, such mask wherein the face mask and the tubes are constructed of polymeric materials known in the art, including without limitation polyethylene, polypropylene, PVC, PVB or other, similar vinyl polymer, synthetic rubber and Bakelite, or any combination thereof.

In another preferred embodiment, such mask wherein the diameter of each reservoir and the diameter of each aperture allow for the snug insertion of tube into aperture with no gap around the rim.

In another preferred embodiment, the mask of the invention wherein the diameter of each tube and aperture is approximately 0.5″ to 2.0″.

In another preferred embodiment, the mask of the invention wherein the diameter of each tube and aperture is approximately 0.75″ to 1.0″.

In another preferred embodiment, the mask of the invention wherein the length of each reservoir tube is approximately 4″ to 8″.

In another preferred embodiment, the mask of the invention wherein the length of each reservoir tube is approximately 6″.

In another preferred embodiment, the mask of the invention wherein the volume of each reservoir tube is approximately 2.5 cubic inches to 3 cubic inches.

In another preferred embodiment, the mask of the invention wherein the volume of each reservoir tube is approximately 2.65 cubic inches.

An oxygen mask of the type comprising a face mask for enclosing the nose and mouth of a person and further comprising the addition of two circular apertures on either side of the nose-covering portion of the mask, and the insertion or attachment of a bendable tube in or over each such aperture; further comprising wherein each such tube is configured in an upward-facing position, such that the tubes produce a capacity to serve as reservoirs for oxygen flowing from the oxygen entry port during times that the patient is not actively inhaling; further comprising wherein the face mask and the tubes are constructed of a pliable material, including without limitation plastic, silicon or rubber, or any combination thereof; further comprising wherein the diameter of each tube and the diameter of each aperture allow for the snug insertion of tube into aperture with no gap around the rim; further comprising a swivel adapter at the top of any attached reservoir bag opening's port to accommodate bag movement if the chest is impeding bag flow; and further comprising wherein the diameter of each tube and aperture is approximately 0.75″ to 1.0″.

An oxygen mask of the type comprising a face mask for enclosing the nose and mouth of a person, further comprising the addition of an aperture on both sides of the mask for the attachment of a nebulizer or a tube attaching a nebulizer.

The mask of claim 16, wherein a one-way valve with a threaded exterior attachment is fitted into the aperture allowing attachment to a nebulizer, or allowing placement of a screw-on cap at times when the nebulizer is not being used.

In another preferred embodiment, the mask of the invention wherein a aperture is cut on either or both sides of the mask for the attachment of a nebulizer or a tube attaching a nebulizer.

In another preferred embodiment, the mask of the invention wherein a one-way valve with a threaded exterior attachment is fitted into the aperture allowing attachment to a nebulizer, or allowing placement of a screw-on cap at times when the nebulizer is not being used.

In another preferred embodiment, the mask of the invention wherein a aperture is cut on both sides of the mask for the attachment of a nebulizer or a tube attaching a nebulizer.

In another preferred embodiment, the mask of the invention wherein a one-way valve with a threaded exterior attachment is fitted into the aperture allowing attachment to a nebulizer, or allowing placement of a screw-on cap at times when the nebulizer is not being used.

In another preferred embodiment, a method of providing extra oxygen capacity to an oxygen mask of the type comprising a face mask for enclosing the nose and mouth of a person by cutting one or more apertures into the side of such mask and attaching an open reservoir to such aperture or apertures.

### BRIEF DESCRIPTION OF THE DRAWINGS

FIG. 1 shows a front view of a standard oxygen face mask with the reservoir tubes attached.

FIG. 2 shows a side view of a standard oxygen face mask with a reservoir tube attached to the near side.

FIG. 3 shows a standard oxygen face mask with reservoir tubes attached fitted to a patient.

FIG. 4 shows a front view of a standard oxygen face mask with reservoir tubes attached, as well as a third, nebulizer valve attached at a lower aspect.

FIG. 5 shows a front view of a standard oxygen face mask with a reservoir bag and reservoir tubes attached, as well as a pair of nebulizer valve attachment ports at a lower aspect.

### DETAILED DESCRIPTION OF THE INVENTION

### Definitions

The following definitions are provided as an aid to understanding the detailed description of the present invention.

The phrase “FIO2”, in the field of medicine, is the fraction of inspired oxygen in a gas mixture. The FIO2 is expressed as a number from 0 (0%) to 1 (100%). The FIO2 of normal room air is 0.21 (21%). A patient's FIO2 may be varied through the use of different oxygen masks, in combination with varying oxygen flow rates. In addition, most oxygen delivery systems have controls for adjusting FIO2. An increased FIO2 is necessary in managing adequate oxygenation in patients who are critically ill due to causes such as major surgery, acute lung injury, sepsis, pneumonia, congestive heart failure, or other cardiopulmonary disease. Generally the FIO2 is maintained at less than 60%. Higher settings can lead to oxygen toxicity.

The phrase “rebreather,” in the field of medicine and more specifically in the field of patient-assisting breathing devices, is where a patient inhales previously exhaled air or gases. In practice this is typically a mask that allows a patient to re-inhale all of their previously exhaled breath. The carbon dioxide from the exhalation advantageously acts to stimulate breathing.

The phrase “partial-rebreather,” in the field of medicine and more specifically in the field of patient-assisting breathing devices, describes where a patient inhales a portion of previously exhaled air along with oxygen or other supplied gases. This is typically an oxygen delivery device in the form of a mask that is configured to allow a patient to re-inhale a portion of their previously exhaled air. Typically, such masks have a soft plastic reservoir bag attached at the end of it that saves one-third of a person's exhaled air, while the rest of the air is allowed to escape the mask via side ports. Any carbon dioxide from the exhalation advantageously acts to stimulate breathing.

The phrase “non-rebreather,” in the field of medicine and more specifically in the field of patient-assisting breathing devices, describes where a patient inhales air or gases that are supplied to the patient and none or almost none of the patient's exhaled air is re-inhaled. This type of mask has a reservoir bag attached, but pressure in the prevents a substantial amount of the exhaled carbon dioxide from getting into the reservoir. A non-rebreather mask allows for more precise control of the patients oxygen intake, which may be up to 100% FIO2.

The term “PaO2,” in the field of medicine, describes the measure of the partial pressure of oxygen in a patient's arterial blood. PaO2 is measured utilizing an Arterial Blood Gas test (ABG) and is a standard means by which a patient's overall oxygen supply may be monitored.

In one embodiment, the invention is comprised of a standard oxygen face mask of the type known in the art, constructed of a pliable material suitable for molding to the general features of the human face and intended to cover the regions of the mouth and nose.

In a preferred embodiment, the invention comprises one circular aperture cut on each side of the nose-covering aspect of the mask for insertion or attachment of a bag reservoir.

In a preferred embodiment, the invention comprises one circular aperture cut on each side of the nose-covering aspect of the mask for insertion or attachment of a reservoir tube. In another preferred embodiment, each such aperture and respective tube has a diameter of between 0.5″ and 2.0″. In a more preferred embodiment, each such aperture and respective tube has a diameter of approximately 0.75″ to 1.0″.

In another preferred embodiment, the tubing used as the reservoir attachment comprises bendable plastic corrugated tubing.

In another preferred embodiment, the tube reservoirs, once attached are molded or bent or upward in a “tusk” or curved shape, allowing oxygen to enter each tube through the aperture and fill from the bottom, and then to empty back into the mask as the patient inhales.

In another preferred embodiment, the tube reservoirs, once attached are molded or bent to form an upward-facing arc.

The size of the oxygen reservoirs can be adjusted according to patient size, lung capacity or one or more spirometry values.

The most common parameters measured in spirometry are Vital capacity (VC), Forced vital capacity (FVC), Forced expiratory volume (FEV) at timed intervals of 0.5, 1.0 (FEV1), 2.0, and 3.0 seconds, Forced expiratory flow 25-75% (FEF 25-75) and Maximal voluntary ventilation (MVV), also known as Maximum breathing capacity. Other tests may be performed in certain situations. Results are usually given in both raw data (litres, litres per second) and percent predicted—the test result as a percent of the “predicted values” for the patients of similar characteristics (height, age, sex, and sometimes race and weight). The interpretation of the results can vary depending on the physician and the source of the predicted values. Generally speaking, results nearest to 100% predicted are the most normal, and results over 80% are often considered normal. However, review by a doctor is necessary for accurate diagnosis of any individual situation.

Forced Vital Capacity (FVC) is the volume of air that can forcibly be blown out after full inspiration, measured in liters. FVC is the most basic maneuver in spirometry tests.

Forced Expiratory Flow (FEF) is the flow (or speed) of air coming out of the lung during the middle portion of a forced expiration. It can be given at discrete times, generally defined by what fraction remains of the functional vital capacity (FVC).

In another preferred embodiment, each tube reservoir is approximately 4″ to 8″ in length. In a more preferred embodiment, the tube reservoirs are approximately 6″ in length.

The formula for determining the volume of a tube with a circular base is it multiplied by base radius squared multiplied by height, shown as:

π×r²×h

In another preferred embodiment, each tube reservoir is approximately 2 cubic inches to 5 cubic inches in volume. In another preferred embodiment, each tube reservoir is approximately 2.65 cubic inches in volume.

In another preferred embodiment, the tube reservoirs are attached to the portion of the mask immediately surrounding each aperture using a commercially available adhesive.

In another preferred embodiment, the base or rim of each tube reservoir is fitted directly into the corresponding aperture and held in place by friction.

Mask and tube construction material can comprise, without limitation, plastic, rubber or silicon. Types of plastic used for mask construction include polyethylene, polypropylene, PVC and other vinyls, PVBs, synthetic rubber and Bakelite, as well as other appropriate polymers known in the art.

In another preferred embodiment, a bagged non-rebreathing-style mask is fitted with side apertures and reservoir tubes as described herein, for additional reservoir capacity and ventilation in addition to the bag itself.

In another preferred embodiment, an oxygen mask is fitted with side apertures and overlaying pockets constructed of polyethylene, polypropylene, PBC or other vinyls, PVBs, synthetic rubber or Bakelite, or other polymers known in the art. Such pockets, comprising a small perforation or plurality thereof, would serve as oxygen reservoirs, filling through the mask aperture and emptying into the mask as the patient inhales.

The mask may be constructed in multiple sizes to fit different sized patients, and also in multiple configurations, depending on factors such as the method of attachment to the patient's head, the method of attachment to the tube or line attached to the oxygen source, etc.

Attachment of the mask to the patient's head may be affected in multiple ways, including without limitation the attachment of one or more bands or straps to encircle the head, or two lateral straps to attach to the patient's ears.

In another embodiment, the standard mask shall have a standard oxygen port for attachment to a tube or line, the distal side of which shall be attached to an oxygen source. Such source shall comprise either an oxygen tank, or a wall attachment in a hospital or clinic with a dedicated oxygen system.

In a preferred embodiment, the standard mask includes one or more circular apertures on the side(s) allowing for attachment of a tube or line connected to a nebulizer for the introduction of nebulized medications into the patient's oxygen flow.

In another preferred embodiment a one-way valve is fitted into each nebulizer aperture.

In another preferred embodiment, the one-way valve fitted into each nebulizer aperture is threaded to allow for attachment of the nebulizer or a screw-on cap when the nebulizer is not in use.

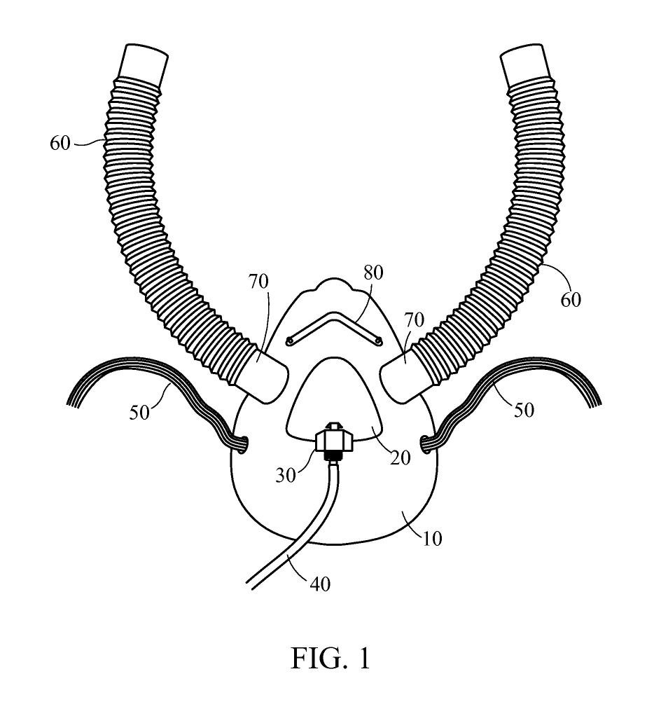

Referring now to the figures, FIG. 1, illustrated line drawing, evidences a front view of facemask 10, comprising nose covering portion 20, into which is fitted oxygen line valve 30, said valve being attached to oxygen line 40. Nosebridge 80 is attached across the upper front of facemask 10, and head strap 50 is attached to each outer side of facemask 10. One aperture 70 is cut on each side of facemask 10, with one tube 60 inserted into each such aperture and is bent at an upward angle.

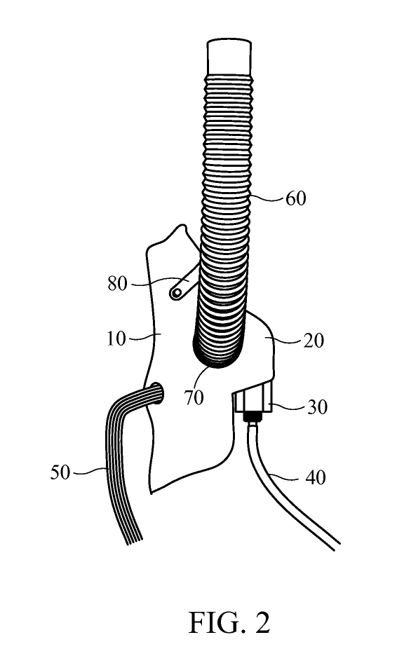

Referring now to FIG. 2, illustrated line drawing, facemask 10 is viewed from the side, with nose covering portion 20 protruding forward, into which is fitted oxygen line valve 30, said valve being attached to oxygen line 40. Nosebridge 80 is attached across the upper front of facemask 10, and head strap 50 is attached to the outer side. Aperture 70 is cut into the side of facemask 10, and tube 60 is inserted into such aperture and is bent at an upward angle.

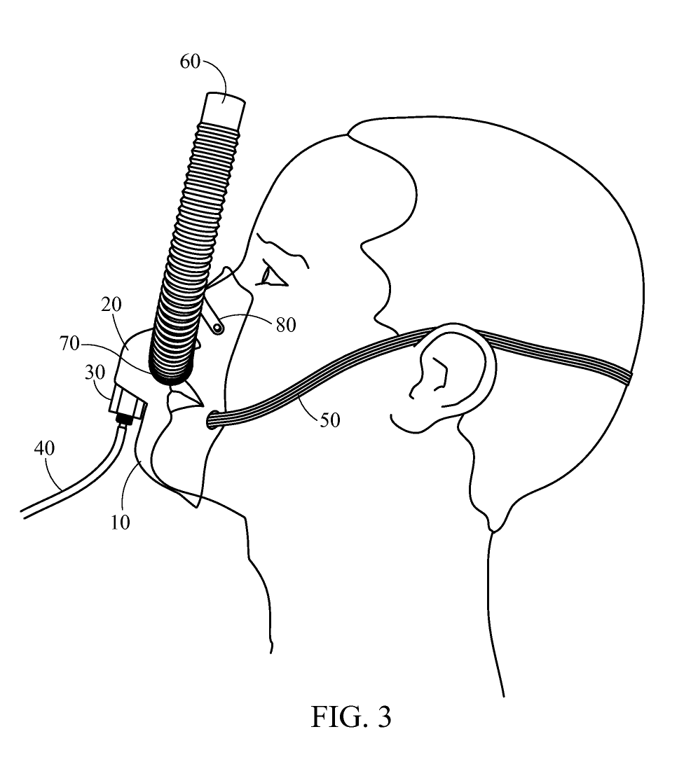

Referring now to FIG. 3, illustrated line drawing, facemask 10 is deployed over a patient's nose and mouth, with nose covering portion 20 located over the patient's nose, with oxygen line valve 30 fitted into such nose covering portion 20 and also attached to oxygen line 40. Nosebridge 80 is attached across the upper front of facemask 10 and located across the bridge of the patient's nose, and head strap 50 is attached to the outer side of facemask 10 and is looped behind the patient's ear to secure the mask in place. Aperture 70 is cut into the side of facemask 10, and tube 60 is inserted into such aperture and is bent at an upward angle.

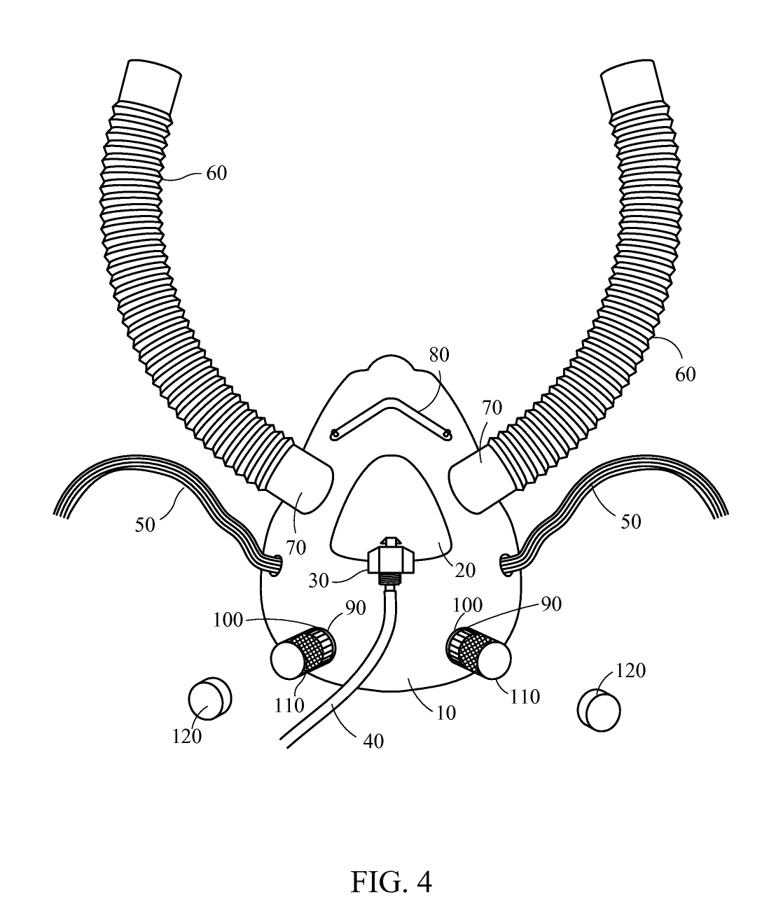

Referring now to FIG. 4, illustrated line drawing, evidences a front view of facemask 10, comprising nose covering portion 20, into which is fitted oxygen line valve 30, said valve being attached to oxygen line 40. Nosebridge 80 is attached across the upper front of facemask 10, and head strap 50 is attached to each outer side of facemask 10. One aperture 70 is cut on each side of facemask 10, with one tube 60 inserted into each such aperture and is bent at an upward angle. A nebulizer aperture 90 is cut into each side of facemask 10 at a lower aspect, with nebulizer valve 100 inserted therein and also attached to nebulizer attachment 110, which can be attached to nebulizer.

Still referring to FIG. 4, Nebulizer valve 100 is preferably a one-way valve, open only when the patient is inhaling and preventing medicine from being wasted while the patient is exhaling. Nebulizer valve 100 is preferably spring loaded to ensure proper functioning in spite of medication residue adhering to or accumulating on the surfaces of the valve. Nebulizer attachment 110 is preferably threaded to allow a nebulizer to be removed when administration of medication is no longer required. The opening of nebulizer attachment 110 can be capped by cap 120 to seal the mask.

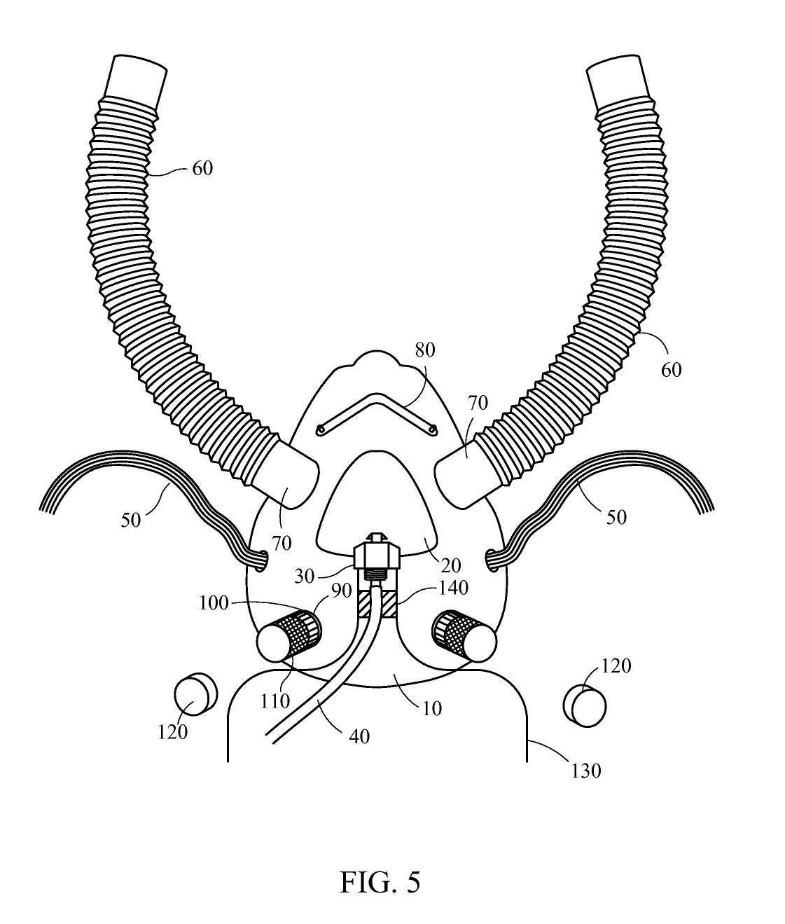

Referring now to FIG. 5, illustrated line drawing, evidences a front view of facemask 10, comprising nose covering portion 20, into which is fitted oxygen line valve 30, said valve being attached to oxygen line 40. Nosebridge 80 is attached across the upper front of facemask 10, and head strap 50 is attached to each outer side of facemask 10. Reservoir bag 130 is attached to the opposite end of oxygen line valve 30. Reservoir bag 130 includes articulating segment 140 allowing reservoir bag 130 to provide unrestricted airflow to mask 10 no matter the orientation od reservoir bag 130. One aperture 70 is cut on each side of facemask 10, with one tube 60 inserted into each such aperture and is bent at an upward angle. A nebulizer aperture 90 is cut into each side of facemask 10 at a lower aspect, with nebulizer valve 100 inserted therein and also attached to nebulizer attachment 110, which can be attached to nebulizer.

The references recited herein are incorporated herein in their entirety, particularly as they relate to teaching the level of ordinary skill in this art and for any disclosure necessary for the commoner understanding of the subject matter of the claimed invention. It will be clear to a person of ordinary skill in the art that the above embodiments may be altered or that insubstantial changes may be made without departing from the scope of the invention. Accordingly, the scope of the invention is determined by the scope of the following claims and their equitable Equivalents.

### Example 1

In this example, the mask is used on a patient suffering from a low blood oxygen level, as determined by measuring the patient's PaO2 level. The patient in this case is breathing spontaneously, but has a low blood oxygen level that needs to be increased to an acceptable level utilizing a single treatment. The mask is placed over the patient's mouth and nose and positioned such that a good seal is achieved between the mask and the patient's skin. Oxygen is then supplied to the mask at a desired flow rate and the reservoir bag is inflated. The flow rate may be adjusted to achieve an oxygen supply of 60% to greater than 80% FIO2. The tubes are adjusted so that they are substantially parallel with the patient's head in order to maintain an optimum oxygen supply to the patient. The patient is then directed to take deep, regular breaths during which the patient's PaO2 level is monitored. Because the mask is able to provide an increased percentage of oxygen to the patient per each breath, the patient's PaO2 level quickly stabilizes to acceptable levels and the mask may be removed more quickly.

### Example 2

In this example, the mask is used to supply FIO2 to a patient requiring a high flow of oxygen in order to maintain required PaO2. The patient in this case is breathing spontaneously, but is unable to maintain appropriate blood oxygen content and sustained supplemental oxygen is required. The mask is placed over the patient's mouth and nose and positioned such that a good seal is achieved between the mask and the patient's skin. Oxygen is then supplied to the mask at a desired flow rate and the reservoir bag is inflated. The flow rate may be adjusted to achieve an oxygen supply of 60% to greater than 80% FIO2. The tubes are adjusted so that they are substantially parallel with the patient's head in order to maintain an optimum oxygen supply to the patient. The patient is then directed to take deep, regular breaths during which the patient's PaO2 level is monitored. Because the mask is able to provide a percentage of oxygen in excess of 80%, the patient's PaO2 level may be maintained at acceptable levels without the need to intubate the patient or utilize a bag valve mask. The patient's PaO2 level is concurrently monitored and the oxygen flow rate adjusted to maintain an acceptable blood oxygenation level.

We claim:

1. An oxygen mask of the type comprising a face mask for enclosing the nose and mouth of a person, comprising the addition of one or two apertures on one or both the sides of the mask, and the insertion or attachment of a reservoir in or over each such aperture, such that the reservoirs produce a capacity to store oxygen flowing from the oxygen entry port during times that the patient is not actively inhaling.

2. The mask of claim 1, wherein the apertures are circular.

3. The mask of claim 1, wherein the apertures are cut into either side of the nose-covering portion of the mask.

4. The mask of claim 1, wherein the reservoirs are tubular in shape with open ends.

5. The mask of claim 4, wherein the reservoir tubes are bendable and configured in an upward-facing position.

6. The mask of claim 1, wherein the face mask and the reservoirs are constructed of a pliable material, including without limitation plastic, silicon or rubber, or any combination thereof.

7. The mask of claim 1, wherein the face mask and the tubes are constructed of polymeric materials known in the art, including without limitation polyethylene, polypropylene, PVC, PVB or other, similar vinyl polymer, synthetic rubber and Bakelite, or any combination thereof.

8. The mask of claim 1, wherein the diameter of each reservoir and the diameter of each aperture allow for the snug insertion of tube into aperture with no gap around the rim.

9. The mask of claim 4, wherein the diameter of each tube and aperture is approximately 0.5″ to 2.0″.

10. The mask of claim 4, wherein the diameter of each tube and aperture is approximately 0.75″ to 1.0″.

11. The mask of claim 4, wherein the length of each reservoir tube is approximately 4″ to 8″.

12. The mask of claim 4, wherein the length of each reservoir tube is approximately 6″.

13. The mask of claim 4, wherein the volume of each reservoir tube is approximately 2.5 cubic inches to 3 cubic inches.

14. The mask of claim 4, wherein the volume of each reservoir tube is approximately 2.65 cubic inches.

15. An oxygen mask of the type comprising a face mask for enclosing the nose and mouth of a person and further comprising the addition of two circular apertures on either side of the nose-covering portion of the mask, and the insertion or attachment of a bendable tube in or over each such aperture; further comprising wherein each such tube is configured in an upward-facing position, such that the tubes produce a capacity to serve as reservoirs for oxygen flowing from the oxygen entry port during times that the patient is not actively inhaling; further comprising wherein the face mask and the tubes are constructed of a pliable material, including without limitation plastic, silicon or rubber, or any combination thereof; further comprising wherein the diameter of each tube and the diameter of each aperture allow for the snug insertion of tube into aperture with no gap around the rim; further comprising a swivel adapter at the top of any attached reservoir bag opening's port to accommodate bag movement if the chest is impeding bag flow; and further comprising wherein the diameter of each tube and aperture is approximately 0.75″ to 1.0″.

16. An oxygen mask of the type comprising a face mask for enclosing the nose and mouth of a person, further comprising the addition of an aperture on both sides of the mask for the attachment of a nebulizer or a tube attaching a nebulizer.

17. The mask of claim 16, wherein a one-way valve with a threaded exterior attachment is fitted into the aperture allowing attachment to a nebulizer, or allowing placement of a screw-on cap at times when the nebulizer is not being used.

18. The mask of claim 1, wherein a aperture is cut on either or both sides of the mask for the attachment of a nebulizer or a tube attaching a nebulizer.

19. The mask of claim 18, wherein a one-way valve with a threaded exterior attachment is fitted into the aperture allowing attachment to a nebulizer, or allowing placement of a screw-on cap at times when the nebulizer is not being used.

20. The mask of claim 15, wherein a aperture is cut on both sides of the mask for the attachment of a nebulizer or a tube attaching a nebulizer.

21. The mask of claim 20, wherein a one-way valve with a threaded exterior attachment is fitted into the aperture allowing attachment to a nebulizer, or allowing placement of a screw-on cap at times when the nebulizer is not being used.

22. A method of providing extra oxygen capacity to an oxygen mask of the type comprising a face mask for enclosing the nose and mouth of a person by cutting one or more apertures into the side of such mask and attaching an open reservoir to such aperture or apertures.

23. The method of claim 22, wherein the size parameters of the open reservoir are based on spirometry values of a patient receiving respiratory therapy including oxygen supplementation.
